(19) United States
(12) Patent Application Publication
(10) Pub. No.: US 2007/0162114 A1
(43) Pub. Date: Jul. 12, 2007

(54) Diaphragm for implantation in the anterior section of a human eye

(76) Inventor: Heino Hermeking, Wuppertal (DE)

Correspondence Address: Thomas M. Galgano, Esq.;GALGANO & ASSOCIATES, PLLC, Suite 204, 20 West Park Avenue, Long Beach, NY, 11561, US

(21) Appl. No.: 10/555,919

(22) PCT Filed: Apr. 30, 2004
(86) PCT No.: PCT/DE04/00906

§ 371 Date: Oct. 5, 2006

(30) Foreign Application Priority Data
May 7, 2003 (DE) 103 20 584.5

(51) Int. Cl. A61F 2/14

(52) U.S. Cl. 623/4.1

(57) ABSTRACT

The diaphragm consists of essentially rigid planar elements that are divided on at least one fold line (14). The division is bridged with an elastic material that enters into an adhesive connection with the planar elements, that allows the diaphragm to be folded in half elastically, and the latter is suitable for unfolding back into its original position by means of its inherent elasticity. Thus, the diaphragm has shape memory and an elastic shape stability.

The invention relates to a diaphragm for implantation in the anterior section of a human eye, for the purpose of creating an artificial pupil opening.

A diaphragm for implantation in the lens capsule bag of an eye is known from WO 98/56 314 A1. The diaphragm is used if aniridia exists, or to take care of iris deficits. Its endocapsulary positioning presupposes the existence of an intact lens capsule bag.

In the case of one variant of WO 98/56 314 A1, there is the possibility of folding the diaphragm. Also, the concept is mentioned of clicking a fixation ring having a circular aperture into the diaphragm, in order to create a precisely circular artificial pupil opening.

A fixation ring for endocapsulary or extracapsulary prosthetic reconstruction in the anterior section region of the human eye is known from the German patent application No. 101 56 463.5, which was published after this application. The fixation ring has an annular body that is divided in the center, and can be folded in half at the division. The body is held together by connecting stirrups.

It is the task of the invention to make available a diaphragm for implantation in the anterior section of a human eye, for the purpose of creating an artificial pupil opening, which can be applied using small-incision surgery, and is characterized by lasting shape stability.

The diaphragm that accomplishes this task consists of essentially rigid planar elements that are divided on at least one fold line. The division is bridged with an elastic material that enters into an adhesive connection with the planar elements, that allows the diaphragm to be folded in half elastically, so that the latter is suitable for unfolding back into its original position by means of its inherent elasticity. Thus, the diaphragm has shape memory and an elastic shape stability.

A preferred embodiment of the diaphragm has at least one straight fold line.

Preferred embodiments of the diaphragm have either a single or two or more parallel fold lines.

In the case of one preferred embodiment, the planar elements of the diaphragm consist of dyed polymethyl methacrylate (PMMA).

In the case of one preferred embodiment, the elastic material that bridges the division of the diaphragm is silicone (polyorganosiloxane), or hydrophilic or hydrophobic acrylate.

In the case of one preferred embodiment, the planar elements of the diaphragm are provided with holes close to the edge, on both sides of the division. These make it possible for the elastic material to penetrate the planar elements, passing through them completely, and to enter into a particularly intimate adhesive bond with the planar elements. The holes are preferably round holes.

In the case of one preferred embodiment, the diaphragm has a central, circular diaphragm opening. The diaphragm can be reinforced with a fixation ring that can be inserted into the diaphragm opening.

In the case of one preferred embodiment, the fixation ring can be folded.

In the case of one preferred embodiment, the fixation ring has a central, circular aperture.

In the case of one preferred embodiment, a lens is provided, which can be clipped into the diaphragm opening of the diaphragm or into the aperture of the fixation ring.

The invention will be explained in greater detail in the following, using exemplary embodiments shown in the drawing. This shows:

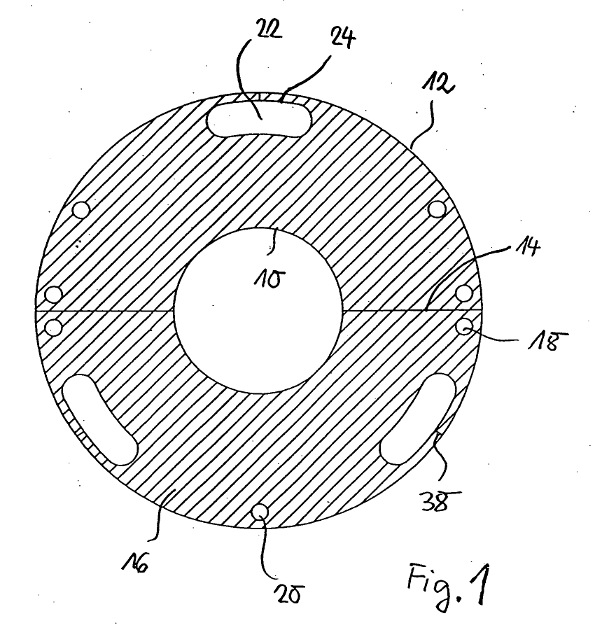

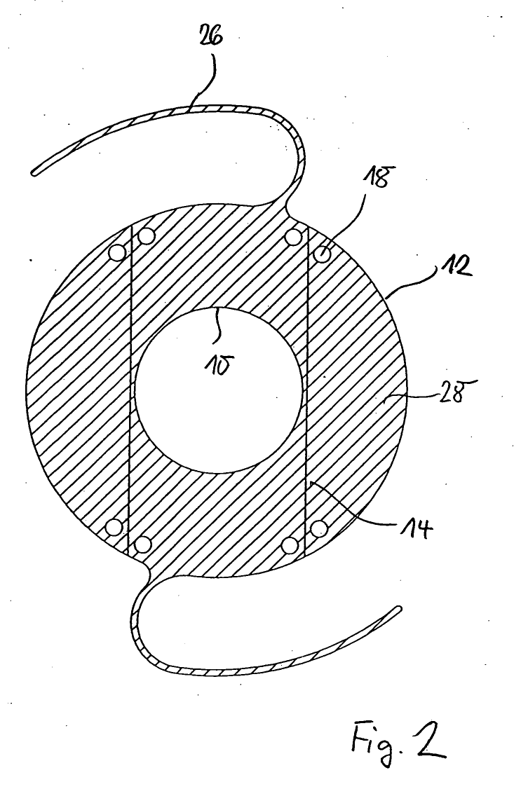

FIG. 1 and FIG. 2 top views of one diaphragm each;

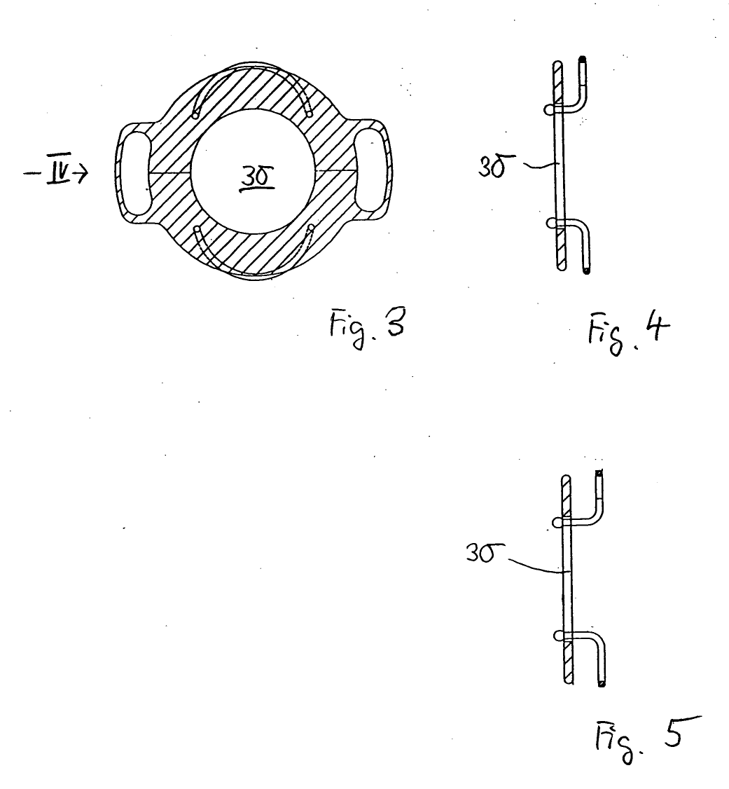

FIG. 3 a top view of a fixation ring;

FIG. 4 a side view of the fixation ring, with a view in the direction IV of FIG. 3;

FIG. 5 a side view of the fixation ring in an alternative embodiment;

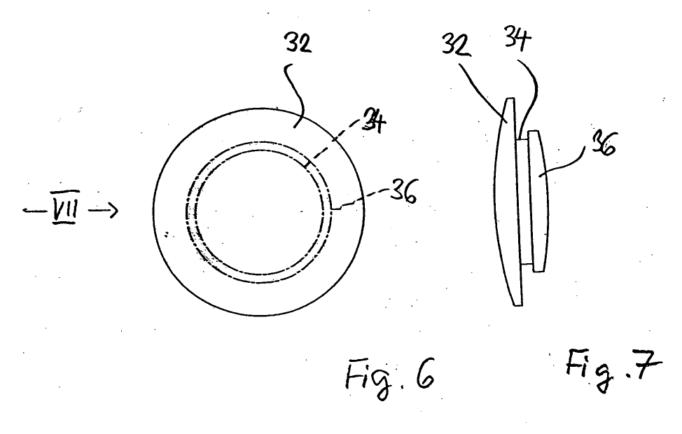

FIG. 6 a top view of a lens; and

FIG. 7 a side view of the lens, with a view in the direction XII of FIG. 6.

The diaphragm shown in FIG. 1 has the basic shape of a flat circular ring having a central circular diaphragm opening and an outer circumference circle 12 that runs concentric to the edge 10 of the opening.

The diaphragm is divided by means of a diametrical fold line 14. The halves 16 are connected at the fold line 14 with an elastic adhesive seam.

The diaphragm is provided with round holes 18 disposed close to its outer edge, in a mirror symmetry arrangement, on both sides of the fold line 14.

These are four round holes 18 having the same size, which lie opposite one another in pairs with reference to the fold line 14. Thanks to the round holes 18, the elastic material of the adhesive seam can penetrate the halves 16.

The diaphragm has three additional round holes 20 having the same size, which lie close to the edge and lie on the same circumferential circle as the round holes 18, on both sides of the fold line 14, offset by 120° from one another. Centered between them, the diaphragm is provided with three significantly larger, essentially oval elongated holes 22 that lie close to the edge. Their long edges 24 are rounded, essentially following the outer periphery of the diaphragm. A central, radial incision 38 extends from the outer long edge 24 to the outer circumference circle 12. The incisions 38 facilitate enclavation of iris tissue. One of the round holes 20 and elongated holes 22 is offset from the fold line 14 by 90°, in each instance.

The diaphragm shown in FIG. 2 has a circular, planar central body having a central, circular diaphragm opening, and an outer circumference circle 12 that runs concentric to the edge 10 of the opening. Two haptic stirrups 26 that lie opposite one another start on the outside of the central body; initially, they are curved forward in the clockwise direction, and during their further progression, they are curved backward parallel to the outer circumference circle 12 of the central body.

The central body of the diaphragm is divided twice, essentially crosswise to the haptic stirrups 26, with two straight fold lines 14. The fold lines 14 extend parallel, at a slight distance from one another, past the diaphragm opening, on both sides. The segments 28 of the central body are connected at the fold lines 14 with elastic adhesive seams.

The diaphragm is provided with round holes 18 that are disposed close to its outer edge, on both sides of the fold lines 14. These are four pairs of round holes 18 that lie on the same circumference circle and are spaced at the same distance from the fold lines 14, in pairs. Thanks to the round holes 18, the elastic material of the adhesive seam can penetrate the segments 28 of the diaphragm.

The diaphragm according to FIG. 2 has dual symmetry of rotation. It makes a transition into itself when turned by 180° about its center point.

The fixation ring according to FIG. 3 to FIG. 5 is the object of the German patent application No. 101 56 463.5, which was published after this application. The fixation ring has a central aperture 30.

The lens shown in FIG. 6 and FIG. 7 can be clipped either into the diaphragm opening of the diaphragms according to FIG. 1 and FIG. 2, or into the central aperture 30 of the fixation ring, depending on its size. The lens has symmetry of rotation relative to a central axis crosswise to its main plane. It has a lens body 32 that is curved convex towards the outside, with a round cylindrical diameter setback 34 behind that. An anchoring part 36 that is also curved convex towards the outside, having a greater diameter, follows adjacent to the latter. The diameter of the lens body 32 is greater than that of the anchoring part 36. After the lens is clipped in, the diameter setback 34 determines the optically exposed lens part.

The invention relates to a foldable diaphragmal iris prosthesis system. The system is created for anatomically pathological situations in which the aim is to produce a diaphragm ring in the anterior section of the eye, to compensate for iris tissue, specifically in those cases where prosthetic positioning in the capsule bag is no longer possible. In this connection, the diaphragmal iris prosthesis system can be sulcus-positioned. Here, although the capsule bag is still present, endocapsulary provision of the prosthesis is no longer possible for various reasons. The capsule merely serves as a support for positioning of the prosthesis in the sulcus ciliaris.

The diaphragm according to FIG. 2 is designed for this location of use. Fundamentally, the diaphragm according to FIG. 1 can also be positioned in this region. However, a prerequisite for fixation of this diaphragm is a residual amount of iris tissue that can allow retroiridal fixation of the prosthesis, for example. Alternatively, the prosthesis is fixed in place by means of a sclera suture. In these cases, the presence of a capsulary support is not necessary, because of the two fixation possibilities just mentioned.

The diaphragm according to FIG. 1 can also be mounted in preiridal manner. In this connection, it is necessary for a residual amount of iris tissue to be present for fixation of the diaphragmal disk, if the disk is supposed to be fixed in preiridal manner. Suture fixation of the disk in the preiridal position is prohibited.

The invention allows intraocular implantation of diaphragmal diaphragm disks into the anterior eye segment, using the technique of small incision surgery, especially if there is no possibility of positioning the prosthesis in endocapsulary manner.

The diaphragmal disks can be folded along fold lines that divide the prosthesis into two or three parts. Cohesion of the prosthesis is guaranteed in that the fold lines are bridged by a silicone layer or adhesive seam. The adhesive consists of the silicone. In the unfolded state, the outside diameter of the diaphragm according to FIG. 1 is approximately 10.5 mm, and in the case of the diaphragm according to FIG. 2, the outside diameter of the central body is approximately 9 mm. In the folded state, the diaphragm according to FIG. 1 can be reduced to a diameter of approximately 5.25 mm, whereby the folded faces have a width of only approximately 3.25 mm, in each instance. This width is the determining factor for the incision size, taking the thickness of the prosthesis (approximately 0.2 mm) into consideration at the same time. In the case of the diaphragm according to FIG. 2, which has two fold lines 14, the width of the central body is reduced from approximately 9 mm to approximately 4.4 mm in the folded state. Taking the prosthesis thickness (approximately 0.2 mm) into consideration added to that, this dimension also determines the required incision size.

The diaphragm according to FIG. 1, for iris enclavation and sclera fixation, is a circular disk having an outside diameter of approximately 10.5 mm. The diaphragm has three elongated holes 22 for iris enclavation. A claw device makes it possible to draw iris tissue into these elongated holes 22 and fix it there. Furthermore, the diaphragmal disk of the diaphragm has round holes 18, 20 that serve to accommodate sutures during sclera fixation. The diaphragm opening of the diaphragmal disk has a diameter of approximately 4 mm. The unfolded state is stabilized by means of the insertion of the fixation ring, which is also foldable, shown in FIG. 3 to FIG. 5, which ring is inserted into the diaphragm with its fold line extending perpendicular to the fold line 14 of the diaphragm. This ensures that the diaphragmal disk remains in the unfolded state and is stabilized there. The diameter of the aperture 30 of the fixation ring is approximately 3 mm. There is the possibility of clipping the lens according to FIG. 6 and FIG. 7 into this aperture 30 of the lens. The lens can be configured according to the individual optical requirements.

The prosthesis according to FIG. 2, for sulcus positioning, has a diaphragmal disk having a diameter of approximately 9.0 mm. To reduce the size during implantation, the disk has two fold lines 14. The fold lines are provided with two round holes 18 at their ends, in each instance. The central part of the diaphragmal prosthesis carries a haptic 26 on the outside, in each instance, the outside diameter of which is approximately 13.75 mm. The haptic 26 serves for positioning in the sulcus ciliaris. The central diaphragm opening of the diaphragm according to FIG. 2 has a diameter of approximately 4 mm. The diaphragm opening can also serve for accommodating the aforementioned fixation ring or a lens that can be clipped into it.

The lens consists of PMMA (polymethyl methacrylate) having pigment dye laid into it, in the colors brown, green, blue, or of dark polycarbonate. These materials are characterized by good, assured biological tolerance (biocompatibility), while greatly limiting light transmission. The front lens body 32 has a diameter of approximately mm, the diameter setback 34 has a diameter of approximately 3 mm, d the rear anchoring part 36 has a diameter of approximately 3.4 mm.

### LIST OF REFERENCE SYMBOLS

10 edge
12 circumference circle
14 fold line
16 half
18 round hole
20 round hole
22 elongated hole
24 longitudinal edge
26 haptic stirrup
28 segment
30 aperture
32 lens body
34 diameter setback
36 anchoring part
38 incision

1. Diaphragm for implantation in the anterior section of a human eye, for the purpose of creating an artificial pupil opening, wherein said diaphragm consists of essentially rigid planar elements that are divided on at least one fold line, and that the division is bridged with an elastic material that enters into an adhesive connection with the planar elements, that allows the diaphragm to be folded in half elastically, and that the latter is suitable for unfolding back into its original position by means of its inherent elasticity.

2. Diaphragm according to claim 1, wherein it has at least one straight fold line.

3. Diaphragm according to claim 1, wherein said diaphragm has a single fold line.

4. Diaphragm according to claim 2, wherein said diaphragm has two or more parallel fold lines.

5. Diaphragm according to, claim 1, wherein said planar elements consist of dyed PMMA (polymethyl methacrylate).

6. Diaphragm according to claim 1, wherein said elastic material is silicone (polyorganosiloxane), or hydrophilic or hydrophobic acrylate.

7. Diaphragm according to claim 1, wherein said planar elements of the diaphragm are provided with holes close to the edge, on both sides of the division.

8. Diaphragm according to claim 7, wherein said holes are round holes.

9. Diaphragm according to claim 1, wherein it has a central, circular diaphragm opening and can be reinforced with a fixation ring that can be inserted into the diaphragm opening.

10. Diaphragm according to claim 9, wherein said fixation ring can be folded.

11. Diaphragm according to claim 9, wherein said fixation ring has a central, circular aperture.

12. Diaphragm according to, claim 1, wherein a lens that can be clipped into the diaphragm opening of the diaphragm or into the aperture of the fixation ring.
